(19) United States
(12) Patent Application Publication
(10) Pub. No.: US 2025/0283141 A1
(43) Pub. Date: Sep. 11, 2025

(54) CULTURE MEDIUM FOR HIGH-THROUGHPUT SEPERATION OF RHIZOBACTERIA FROM CROPS, PREPARATION METHOD AND APPLICATION THEREOF

(71) Applicant: ECOLOGY INSTITUTE OF SHANDONG ACADEMY OF SCIENCES THE SINO JAPANESE FRIENDSHIP BIOTECHNOLOGY RES CEN, Jinan (CN)

(72) Inventors: Xinjian Zhang, Jinan (CN); Fangyuan Zhou, Jinan (CN); Xiaoqing Wu, Jinan (CN); Susu Fan, Jinan (CN); Guangzhi Zhang, Jinan (CN); Xueying Xie, Jinan (CN)

(21) Appl. No.: 19/212,678

(22) Filed: May 20, 2025

Related U.S. Application Data
(63) Continuation of application No. PCT/CN2024/087194, filed on Apr. 11, 2024.

(30) Foreign Application Priority Data
Jul. 12, 2023 (CN) 202310848634.1

(51) Int. Cl. C12Q 1/04; C12M 1/12; C12N 1/20; C12Q 1/6869; C12Q 1/689

(52) U.S. Cl. CPC C12Q 1/045; C12M 37/02; C12N 1/205; C12Q 1/6869; C12Q 1/689

(57) ABSTRACT

The present invention relates to the technical field of the environmental microbiology, especially to culture medium for high-throughput separation of rhizobacteria from crops, preparation method and application thereof. The culture medium for high-throughput separation of crop rhizobacteria comprises a 1/l0 TSB medium and a culture medium additive (solution), wherein culture medium additive (solution) is obtained by mixing the crop plants with TSB medium and then by crushing, fermenting, filtering as well as sterilizing. By utilizing the interaction network between multiple microorganisms, crop-source nutrients are fermented using microbiome in-situ in the rhizosphere, and the fermented products are used as carbon and nitrogen source for separation of rhizosphere microorganisms, which can simulate the in-situ environment of rhizosphere microorganisms to the maximum extent, thereby obtaining more microbial species. Combined with the high-throughput separation method, culture medium provided by present invention can obtain more than 60% of bacterial species obtained by high-throughput sequencing.

### TECHNICAL FIELD

The present invention relates to technical field of environmental microbiology, especially to culture medium for high-throughput separation of rhizobacteria from crops, preparation method and application thereof.

### TECHNICAL BACKGROUND

The crop rhizosphere provides an important habitat for many microorganisms. Particularly, the secretions produced by the roots can serve as carbon and nitrogen sources for a variety of the microorganisms. Therefore, microorganisms such as filamentous fungi, yeasts, actinomycetes and bacteria are enriched in the crop rhizosphere. These microorganisms are collectively referred to as rhizosphere microbiome which plays an important role in promoting nutrient absorption by crops, improving stress resistance of crops and promoting increased production of crops.

In recent years, with development of high-throughput sequencing technology, researchers get a more thorough understanding of diversity of rhizosphere microorganisms and have a deeper understanding of the functions of the rhizosphere microbiome, especially relying on metagenomics technology. As an important resource library of the probiotic strains for the crops, more and more researchers also rely on high-throughput sequencing data to separate the rhizobacteria from crops, hoping to screen out beneficial strains that can be used for field planting to decrease occurrence of pests as well as diseases and to increase crop production. The most widely used high-throughput method is rhizobacteria separation method based on limiting dilution developed by LIU Yongxin et al., and Chinese patent CN111518729A discloses a high-throughput separation and cultivation method for root microbiome of corps, which can separate and cultivate a large number of bacteria in a short time with less manpower, can quickly identify types of bacteria via one-click sequence analysis process and can assemble the root microbiome of corps, thus providing technical support for obtaining more resources on root microorganisms of corps. However, rice root bacteria isolated and cultured by existing method only account for about 60% of total number of species of rice root bacteria. Besides, Arabidopsis root bacteria isolated and cultured by existing method only account for about 58% of total number of species of Arabidopsis root bacteria. Therefore, to increase the number of isolated species of rhizobacteria, it is urgent to develop new culture media, thus meeting requirements of high-throughput separation of rhizosphere microorganisms.

### SUMMARY OF THE INVENTION

To increase the number of species of rhizosphere microorganisms of crops obtained using high-throughput separation and cultivation method, the present invention provides culture medium for high-throughput separation of the rhizobacteria from crops, preparation method and application thereof.

The present invention provides a culture medium for high-throughput separation of crop rhizobacteria, comprising a 1/10 TSB medium and a culture medium additive (solution), wherein culture medium additive (solution) is obtained by mixing crop plants with TSB medium and then by crushing, fermenting, filtering and sterilizing, and wherein the crop is cucumber.

Furthermore, mass ratio of 1/10 TSB medium to culture medium additive (solution) is 1:1.

The present invention also provides a method for preparing the above-mentioned culture medium, comprising preparing an agar aqueous solution and sterilizing it at high temperature, then mixing 1/10 TSB culture medium, culture medium additive (solution) and agar aqueous solution to obtain final agar concentration of 1.2%, and preparing a culture medium plate after condensation.

Furthermore, the method for preparing the culture medium additive (solution) specifically comprises:
mixing the whole plant of the crop to be separated with 1/10 TSB medium at a mass ratio of 1:1 to 1:10 and crushing it, then fermenting it at 4° C. to 25° C. for 12 hours, and then filtering as well as sterilizing the fermented product to finally obtain it.

Furthermore, different types of the antibiotics are added to the culture medium according to conventional methods to inhibit growth of non-target microbiome. Commonly used antibiotics comprise nystatin, cycloheximide, penicillin, tetracycline, streptomycin sulfate, ampicillin, etc.

The present invention also provides an application of culture medium in high-throughput separation and cultivation of crop rhizosphere microorganisms.

Furthermore, the samples of the crop root are ground under sterile conditions to obtain a homogenate, then the homogenate is fully diluted with the above-mentioned culture medium and transferred to a 96-well plate for culturing until the number of turbid wells in the 96-well plate is no longer increased, then the genomic DNA of microorganisms in the turbid wells is extracted for high-throughput sequencing analysis, and finally the crop root microbiome can be obtained.

Furthermore, the dilution factor is such that the number of turbid wells in 96-well plate is ultimately 30 to 35.

Furthermore, specific operation of homogenate is as follows: collecting samples of crop root and rinsing the roots with the sterile water, then transferring the roots to a new centrifuge tube and adding 30 mL of 10 mM magnesium chloride solution; placing the centrifuge tube on a shaker and washing it three times, and absorbing liquid on roots with sterile filter paper after being taken out; cutting roots into pieces with sterile scissors and mixing them evenly, then weighing 0.02 g of root tissue and putting it into a 1.5 mL centrifuge tube; adding 1 mL of the sterile magnesium chloride solution to 1.5 mL centrifuge tube, wherein 500 μL of the magnesium chloride solution is used to grind the roots and 500 μL of magnesium chloride solution is used to rinse the grinding rod, and then grinding the root tissue into a homogenate; transferring the homogenate to a 50 mL centrifuge tube containing 25 mL of the 10 mM magnesium chloride, mixing it well and setting it aside.

Furthermore, culture medium additive (solution) is prepared by mixing 100 mL of 1/10 TSB medium with 65 g of cucumber seedlings with roots, which were fully crushed, fermented at 10° C. for 12 h and filtered for sterilization.

The beneficial effects of the present invention are as follows:

The culture medium for high-throughput separation of crop rhizosphere bacteria provided by the present invention is based on theory of multi-species interaction in rhizosphere microbiome. By utilizing interaction network between multiple microorganisms, the crop-source nutrients are fermented using the microbiome in-situ in the rhizosphere, and the fermented products are used as carbon source as well as nitrogen source for separation of rhizosphere microorganisms, which can simulate the in-situ environment of the rhizosphere microorganisms to maximum extent, thereby obtaining more microbial species.

Compared with existing culture media, culture medium provided by the present invention can obtain more types of the bacterial species. Combined with high-throughput separation method, more than 60% of bacterial species can be obtained through the high-throughput sequencing, while conventional separation and cultivation method can only obtain 10% to 15% of bacterial species.

### DESCRIPTION OF DRAWINGS

To more clearly illustrate technical solutions in embodiments or prior art of the present invention, the accompanying drawings to be used in description of embodiments or prior art will be briefly described below. Obviously, accompanying drawings in following description are only some of embodiments of the present invention, and other accompanying drawings may be obtained based on these drawings by a person of ordinary skill in the art without creative labor.

FIG. 1 is digital photos showing colony growth of cucumber rhizobacteria separated using different culture medium plates in Test Embodiment 1 of the present invention.

FIG. 2A is the result of colony count of cucumber rhizobacteria separated using different culture medium plates;

FIG. 2B is result of species number of cucumber rhizobacteria separated using the same culture medium plate. In the box plots, those with the same labeled letters are not significantly different, while those with different labeled letters are significantly different.

FIG. 3 is a community composition diagram obtained by collecting the plate colonies for high-throughput sequencing in Test Embodiment 1 of the present invention.

FIG. 4 is a diagram showing genus-level quantitative results of cucumber rhizobacteria obtained by high-throughput separation and cultivation in Test Embodiment 2 of present invention.

### DETAILED DESCRIPTION OF THE EMBODIMENTS

To enable those skilled in the art to better understand the technical solutions in the present invention, the technical solutions in the embodiments of the present invention will be clearly and completely described below in conjunction with the attached drawings in the embodiments of the present invention. Obviously, the embodiments described are only part of the embodiments of the present invention, not all embodiments. Based on embodiments in the present invention, all other embodiments obtained by those skilled in prior art without creative work belong to claimed scope of the present invention.

### Embodiment 1

### 1. Preparation of Culture Medium Additive (Solution)

First, the cucumber seedlings with roots are collected, the soil on the roots is shaken off, and then the roots are rinsed with sterile water to wash off attached soil. 50 g of washed cucumber seedlings with the roots are mixed with 100 mL of the 1/10 TSB medium, crushed thoroughly, and fermented at 10° C. for 12 h. The fermentation liquid is filtered using gauze folded to 7 layers, then filtered three times using a single-layer filter paper. Finally, filtrate is filtered and sterilized using a 0.22 μm sterile filter to obtain the culture medium additive A.

### 2. Preparation of Culture Medium

The agar aqueous solution is prepared and sterilized at high temperature, then 1/10 TSB medium, culture medium additive (solution) as well as agar aqueous solution are mixed to obtain final agar concentration of 1.2%, and culture medium plate A is prepared after condensation.

### Embodiment 2 to 6

The present embodiments are different from Embodiment 1 in that weight of cucumber seedlings with roots, the fermentation conditions and the fermentation time used in embodiments 2 to 6 are as shown in Table 1, and the other treatments are the same as in Embodiment 1 to prepare culture medium additives B to F and then to prepare culture medium plates B to F.

TABLE 1
Amounts of Raw Materials Used in Culture Medium Additives
and Fermentation Conditions in Embodiments 1 to 6

| | | | Weight of | Fermen- | |
|---|---|---|---|---|---|
| | | | Cucumber | tation | |
| | Culture | 1/10 TSB | Seedlings | Temper- | Fermen- |
| | Medium | Medium | with Roots | ature | tation |
| Embodiment | Additive | (mL) | (g) | (° C.) | Time (h) |
| 1 | A | 100 | 50 | 10 | 12 |
| 2 | B | 100 | 50 | 4 | 12 |
| 3 | C | 100 | 50 | 25 | 12 |
| 4 | D | 100 | 65 | 10 | 12 |
| 5 | E | 100 | 120 | 10 | 12 |
| 6 | F | 100 | 25 | 10 | 12 |

### Test Embodiment 1: Separation and Cultivation Test of Cucumber Rhizobacteria

1) The test requires a total of 8 culture medium plates, wherein one is a 1/10 LBA medium plate, wherein one is a 1/10 TSA medium plate with agar concentration of 1.2% and wherein the remaining 6 culture media are culture medium plates A to F prepared in Embodiments 1 to 6.

2) Sampling: Cucumber plants that had been colonized for 60 days are collected in a solar greenhouse. After the collection, the above-ground parts of plants are cut off, soil attached to the roots is shaken off, and 10 g of main roots, lateral roots and adventitious roots are cut into sterile 50 mL centrifuge tubes. After each plant is collected, the scissors are disinfected with 75% ethanol and rinsed with sterile water three times before use. After collecting, centrifuge tubes containing samples are placed in an ice box and transported to the laboratory. 35 mL of sterile 1×PBS buffer is added to each sample tube, and the tube is placed in a shaker at 180 r/min for 20 minutes. After that, the moisture on root surface is absorbed with sterile filter paper, and washing is repeated three times.

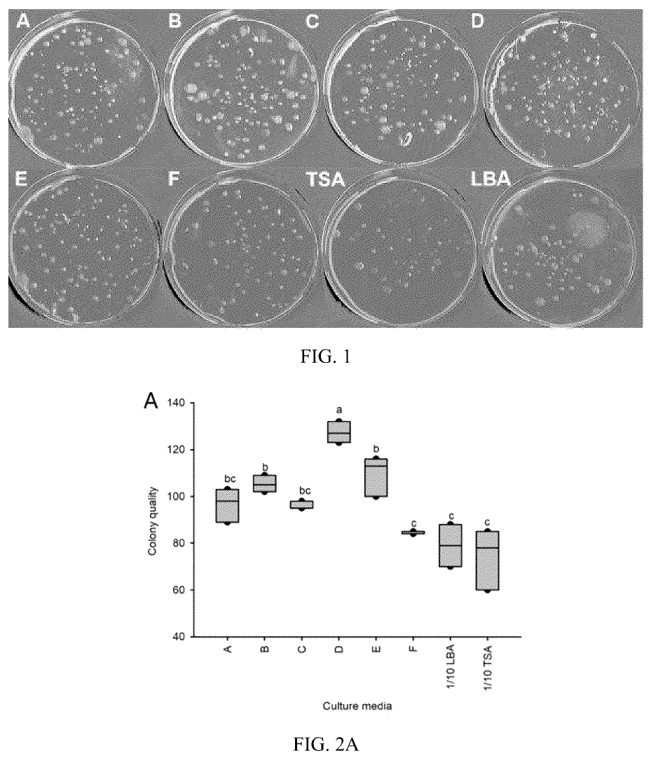

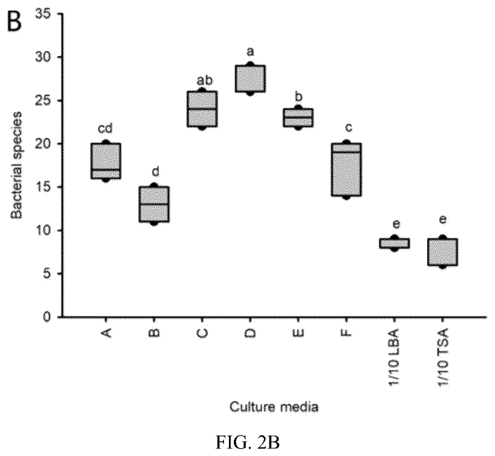

3) Separation and cultivation: 0.02 g of cleaned root sample is accurately weighed, 1 mL of sterilized 1×PBS buffer is added and fully ground, and then 1×PBS buffer is used to dilute the sample in a 10-fold gradient to 10⁻¹, 10⁻², 10⁻³, 10⁻⁴, 10⁻⁵, 10⁻⁶, 10⁻⁷ as well as 10⁻⁸. 25 μL of each concentration is pipetted with pipette and dropped onto 8 culture medium plates. The samples are spread evenly at once with sterile triangular glass coating rod, and after bacterial solution is fully absorbed, the plate is sealed with a sealing film. After inverting and culturing at 28° C. for 36 h, the number of colonies on each plate is counted. 6 plates are plated with each medium at each dilution concentration. Among them, under the condition of 1000-fold dilution, the growth of colonies on 8 culture medium plates is shown in FIG. 1. The colonies grown on culture medium plates A to F are significantly more than those on 1/10 LBA medium and 1/10 TSA medium. The results of the plate counts of the 1000-fold dilution are shown in Table 2 and FIG. 2A. It can be observed that the number of colonies grown in different culture media is significantly different (F_{7.16}=15.247, p<0.001), among which the number of colonies grown on culture medium plate D is the largest.

TABLE 2
Number of Colonies Grown on Different Culture Medium Plates

| | Culture Medium | | | | | | | |
|---|---|---|---|---|---|---|---|---|
| | A | B | C | D | E | F | 1/10 LBA | 1/10 TSA |
| Number | 96.6 ± 7.1 | 105.3 ± 3.5 | 97.0 ± 1.7 | 127.3 ± 4.5 | 113.0 ± 13.0 | 84.6 ± 0.5 | 79.0 ± 9.0 | 74.3 ± 12.8 |
| of | | | | | | | | |
| Colonies | | | | | | | | |

4) Species identification: Colonies with the different colors, sizes, thickness, transparency and texture are selected, purified and cultured on TSA medium using the three-zone line-drawing method with an inoculation loop, and after obtaining the strains, the line-drawing is repeated three times to obtain pure strain. The strains are grouped according to their morphology. Representative strains are selected and inoculated into the test tubes containing 2 mL of liquid TSB medium and cultured at 28° C. and 180 r/min for 12 h. After that, 1 mL of bacterial solution is transferred to a 2 mL centrifuge tube, and the bacteria are collected after centrifugation at 13000 r/min for 2 min. DNA is extracted using a bacterial genome extraction kit, and then the bacterial 16S rRNA gene is amplified using 27F as well as 1492R. PCR reaction system (25 μL): 1 μL DNA (20 ng), 0.5 μL primers each, 12.5 μL 2×Taq PCR Buffer and 10.5 μL ddH2O. PCR amplification program: 95° C. for 5 min; 94° C. for 30 s, 52° C. for 30 s, 72° C. for 90 s, the above are cycled 35 times, and finally at 72° C. for 10 min. The PCR products are detected by 1.2% of agarose gel electrophoresis, and the samples with a fragment size of about 1500 bp are sent to Sangon Biotech (Shanghai) Co., Ltd. for sequencing using Roche 454 platform. The obtained sequences are compared with the closely related strains in GenBank for bacterial species identification, and the species of bacteria separated from each plate are counted. Results are shown in Table 3 and FIG. 2B. The most bacterial species grow on culture medium plate D.

TABLE 3
Number of Bacterial Species Grown on Different Culture Medium Plates

| | Culture Medium | | | | | | | |
|---|---|---|---|---|---|---|---|---|
| | A | B | C | D | E | F | 1/10 LBA | 1/10 TSA |
| Number | 17.6 ± 2.1 | 13.0 ± 2.0 | 24.0 ± 2.0 | 28.0 ± 1.7 | 23.0 ± 1.0 | 17.6 ± 3.2 | 8.6 ± 0.5 | 7.0 ± 1.7 |
| of | | | | | | | | |
| Bacterial | | | | | | | | |
| Species | | | | | | | | |

### 5) High-Throughput Amplicon Sequencing Detection

Culture medium plate D and 1/10 TSA medium plates with colonies diluted according to all dilution factor are rinsed thoroughly with 3 mL of sterile water, then bacteria on the surface are collected and mixed, and then the total DNA is extracted. The V5 to V7 areas of the 16S rRNA of rhizobacteria are amplified using the specific primers 799F and 1193R, and the barcode sequence (provided by Shanghai Meiji Biomedical Co., Ltd.) is added to the 5′ end of the PCR product. PCR reaction system (50 μL): 5 μL DNA template (10 ng), 1.5 μL primers each, 5 μL 2 mmol/L dNTPs, 2 μL MgSO₄, 5 μL 10×KOD Buffer, 1 μL KOD Plus, and ddH₂O is made up to 50 μL. The PCR amplification program: 94° C. for 2 min; 94° C. for 30 s, 63° C. for 30 s, 68° C. for 30 s, the above are cycled 30 times, and finally at 68° C. for 5 min.

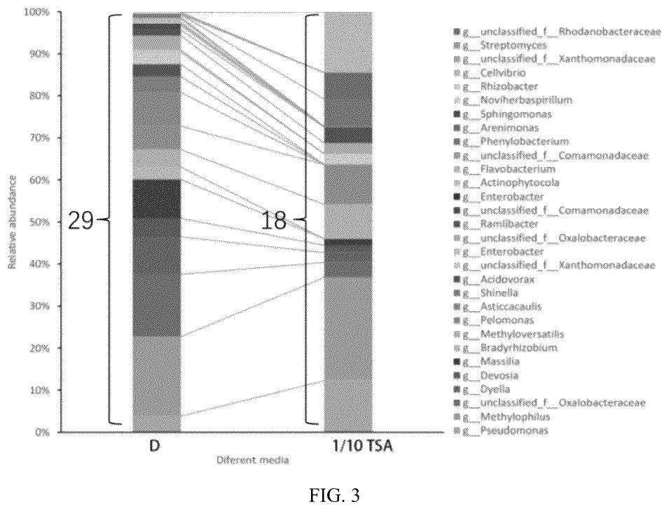

Each sample is repeated three times, and the three PCR products are mixed and detected by 2% agarose gel electrophoresis. The PCR product is recovered by gel excision using AxyPrep DNA gel recovery kit and sent to Shanghai Meiji Biomedical Co., Ltd. for library construction and sequencing. The sequencing platform is Illumina Miseq PE250. The sequencing data are analyzed using the “Bioinformatics Cloud” platform (www.majorbio.com) of Shanghai Meiji Biomedical Co., Ltd. In the cloud platform, the QIIME v.1.9.1 (Quantitative Insights Into Microbial Ecology) software package is used for quality control and data analysis of the sequencing data. Before data processing, sequences with a length of less than 200 bp, containing ambiguous bases, and primer mismatches of more than 2 bases are removed from the sequencing data. The screened sequences are clustered into OTUs using UPARS in the USEARCH software package at a similarity threshold of 97%. The sequence with the highest abundance is selected as representative sequence of OTUs and taxonomic analysis is performed using RDP classifier v.2.2. SILVA database (http://www.arb-silva.de) is used to set the confidence threshold to 70% for the taxonomic annotation, and the OTU composition as well as read counts of each sample at different taxonomic levels are counted. Due to limitations of sequencing platform and annotation database, some sequences in the present test could not be annotated to the species. Therefore, FastTree 2.1.3 is used to construct the maximum likelihood tree of species composition at the genus level based on the information of each sequence, and the read counts at the genus level are counted as species abundance. The results are shown in FIG. 3. A total of 29 genera of bacteria are detected on the culture medium plate D, while a total of 18 genera of bacteria are detected on the 1/10 TSA medium plate.

### Test Embodiment 2: High-Throughput Separation, Cultivation and Identification of Cucumber Root Bacteria

Samples of cucumber roots are collected, and the roots are rinsed with sterile water, then roots are transferred to a new centrifuge tube and 30 mL of 10 mM magnesium chloride solution is added. Centrifuge tube is placed on a shaker, washed repeatedly 3 times, and the liquid on roots is absorbed with sterile filter paper after being taken out. The roots are cut into pieces with sterile scissors and mixed evenly, and 0.02 g of root tissue is weighed and placed in a 1.5 mL centrifuge tube. 1 mL of sterile magnesium chloride solution is added to the 1.5 mL centrifuge tube, 500 μL of magnesium chloride solution is used to grind roots, and 500 μL of magnesium chloride solution is used to rinse the grinding rod and grind to a homogenate. The homogenate is transferred to a 50 mL centrifuge tube containing 25 mL of 10 mM magnesium chloride, mixed and set aside.

There are three groups in the present test, namely control group 1, control group 2 as well as test group. Each group possesses 3 replicates, and the average value is taken. The treatment of each group is as follows:

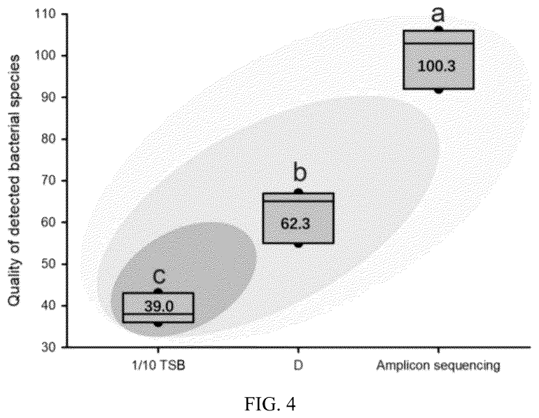

Control group 1: The total DNA in the homogenate is extracted, and then the number of bacterial species in the sample cucumber rhizosphere is detected according to the “high-throughput amplicon sequencing detection” method in Test Embodiment 1, which served as Control Group 1 (i.e., Amplicon sequencing in FIG. 4).

Control group 2: Homogenate is fully diluted with 1/10 TSB medium and then transferred to a 96-well plate for culturing until number of turbid wells in 96-well plate is no longer increased. The dilution factor is such that the number of turbid wells in 96-well plate is ultimately 30 to 35. The genomic DNA of microorganisms in the turbid wells is extracted, and double-sided tag PCR amplification method is used to identify 16S rRNA gene of separated and cultured bacteria with high throughput to obtain the cucumber root microbiome (i.e., 1/10 TSB in FIG. 4).

Test group: The homogenate is fully diluted using the culture medium plate D prepared in Embodiment 4 and then transferred to a 96-well plate for culturing until the number of turbid wells in the 96-well plate is no longer increased, and dilution factor is such that number of turbid wells in 96-well plate is ultimately 30 to 35. The genomic DNA of microorganisms in turbid wells is extracted, and double-sided tag PCR amplification method is used to identify 16S rRNA gene of separated and cultured bacteria with high throughput to obtain the cucumber root microbiome (i.e., D in FIG. 4).

The high-throughput separation of cucumber rhizobacteria and the test method of control group 2 and the test group refer to the method of LIU Yongxin et al (Zhang, J., Liu, Y.-X., Guo, X., Qin, Y., Garrido-Oter, R., Schulze-Lefert, P. and Bai, Y. (2021). High-throughput cultivation and identification of bacteria from the plant root microbiota .Nat Protoc16(2): 988-1012.)

The results of the above three groups are shown in FIG. 4. At the genus level, a total of 100.3 genera of bacteria are detected using the high-throughput amplicon sequencing method, 39.0 genera of bacteria are separated using 1/10 TSB medium, and 62.3 genera of bacteria are separated using culture medium plate D prepared in Embodiment 4. The number of bacterial species detected by three methods is significantly different (F_{2.6}=79.34, p<0.001). The high-throughput amplicon sequencing method can detect almost all bacterial species in the sample, but pure strains cannot be obtained. The number of species separated using the gradient dilution method combined with 1/10 TSB medium is significantly lower than that of technical solution of the present invention, while number of bacterial species that can be separated by technical solution of the present invention is higher as 59.7%.

Although the present invention has been described in detail by referring to accompanying drawings and in combination with the preferred embodiments, the present invention is not limited thereto. Without departing from the spirit and essence of the present invention, a person of ordinary skill in the art may make various equivalent modifications or substitutions to the embodiments of the present invention, and these modifications or substitutions shall be within scope of the present invention. Any person of ordinary skill in the prior art may easily think of changes or substitutions within the technical scope disclosed by the present invention, and these shall be within the scope of protection of the present invention.

What is claimed is:

1. A culture medium for high-throughput separation of crop rhizobacteria, comprising a 1/10 TSB medium and a culture medium additive (solution), wherein the culture medium additive (solution) is obtained by mixing the crop plants with TSB medium and then by crushing, fermenting, filtering and sterilizing, and wherein the crop is cucumber.

2. The culture medium according to claim 1, wherein mass ratio of 1/10 TSB medium to culture medium additive (solution) is 1:1.

3. A method for preparing the above-mentioned culture medium according to claim 1, comprising preparing an agar aqueous solution and sterilizing it at high temperature, then mixing the 1/10 TSB culture medium, culture medium additive (solution) and agar aqueous solution to obtain final agar concentration of 1.2%, and preparing a culture medium plate after condensation.

4. The preparation method according to claim 3, wherein the method for preparing culture medium additive (solution) specifically comprises:
Mixing the whole plant of the crop to be separated with the 1/10 TSB medium at a mass ratio of 1:1 to 1:10 and crushing it, then fermenting it at 4° C. to 25° C. for 12 hours, and then filtering and sterilizing the fermented product to finally obtain it.

5. The preparation method according to claim 3, wherein antibiotics are added to culture medium to inhibit growth of non-target microbiome.

6. An application of culture medium in high-throughput separation as well as cultivation of crop rhizosphere microorganisms according to claim 1.

7. The application according to claim 6, wherein samples of the crop root are ground under sterile conditions to obtain a homogenate, then the homogenate is fully diluted with the above-mentioned culture medium and transferred to a 96-well plate for culturing until the number of turbid wells in 96-well plate is no longer increased, then the genomic DNA of microorganisms in the turbid wells is extracted for high-throughput sequencing analysis, and finally the crop root microbiome can be obtained.

8. The application according to claim 7, wherein the dilution factor is such that number of turbid wells in 96-well plate is ultimately 30 to 35.

9. The application according to claim 7, wherein culture medium additive (solution) is prepared by mixing 100 mL of 1/10 TSB medium with 65 g of cucumber seedlings with roots, which were fully crushed, fermented at 10° C. for 12 h and filtered for sterilization.
